(19) United States
(12) Patent Application Publication
(10) Pub. No.: US 2005/0008574 A1
(43) Pub. Date: Jan. 13, 2005

(54) Use of intravenous contrast media for projection mammography

(76) Inventors: Ulrich Speck, Berlin (DE); Irtel Von Brenndorff, Braunschweig (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/851,213

(22) Filed: May 24, 2004

Related U.S. Application Data
(63) Continuation of application No. 09/446,328, filed on Apr. 17, 2000, now abandoned.

(30) Foreign Application Priority Data
Jun. 20, 1997 (EP) 97250190.2

(51) Int. Cl. A61K 49/04

(52) U.S. Cl. 424/9.42

(57) ABSTRACT

The invention relates to the use of intravenous contrast media for projection mammography as well as new devices for projection mammography. The invention therefore relates to the use of intravenous contrast media for the production of a diagnostic agent for projection mammography. Through the additional intravenous administration of contrast media, projection mammography achieves a sensitivity that is comparable to that of the most modern processes such as magnetic resonance tomography (MRT) while being considerably more versatile and avoiding the costs of MRT. The new process can be implemented simply and without special stress on the patients and provides a significant improvement in a) sensitivity to the detection of focal lesions in the breast, and b) additional information on the nature of lesions detected previously.

The invention relates to the use of intravenous contrast media for projection mammography as well as new devices for projection mammography.

### PRIOR ART

For a decade, mammography has been an established and steadily improved x-ray technique for early detection, radiologic identification, characterization, and localization of mammary tumors. In many respects, it is unparalleled in its performance and availability to patients. The greatest drawback is its imperfect detection sensitivity for tumors that are small and without detectable microlime.

Early on, attempts were made to use contrast media to improve projection mammography. For this purpose, suitable preparations were introduced into the milk ducts, and their dispersion into the breast was used for detecting and characterizing lesions. The work of R. Bjrn-Hansen provides a survey: Contrast-Mammography, Brit. J. Radiol. 38, 947-951, 1965. The technique is also known as galactography. The contrast is achieved by concentrated iodine-containing contrast media (>100 mg of iodine/ml). In addition, contrast media were injected directly into suspicious or tumorous lesions of the breast either to characterize the latter (e.g., Lehto, M. and Mathiesen, T. I.: Adenography: An Ancillary Diagnostic Method of Circumscribed Lesions of the Breast with a Positive Contrast Agent, Breast Dis, 6, 259-268, 1993) or to label the latter (e.g., Raininko, R.; Linna, M. I.; Rasanen, O: Preoperative Localization of Nonpalpable Breast Tumors. Acta. Chir. Scand, 142, 575-578, 1976). In both cases, undiluted, commercially available contrast media are used directly for visualization.

The intravenous administration of x-ray contrast media for visualization of parenchymatous processes in projection radiography is the very rare exception. It is successful only if the contrast medium actively accumulates in a tissue or organ. In this respect, there are to date two examples: The visualization of the healthy renal parenchyma by the now commonly used urographic agents and the visualization of the healthy liver and spleen parenchyma by emulsions or suspensions of x-ray-opaque substances. Both methods are no longer used (liver, spleen) or are used only in exceptional cases (kidney). It has never been possible to use intravenously administered x-ray contrast media for direct contrasting of tumors- of relevant size in projection radiography.

Computer tomography and especially magnetic resonance tomography are known for their very much higher measuring sensitivity for contrast media. It was still a surprise, however, that both techniques made it possible to detect mammary tumors with great reliability after intravenous contrast medium injection (Gisvold, J. J.; Karsell, P. R.; Reese, E. C.: Clinical Evaluation of Computerized Tomographic Mammography. Mayo Clin Proc 52, 181-185, 1977; Teifke, A.; Schweden, F.; Cagil, H.; Kanczor, H. U; Mohr, W.; Thelen, M.: Spiral-Computertomographie der Mamma [Spiral Computer Tomography of the Breast]. Fortschr. Röntgenstr 161, 495-500, 1994; Heywang, S. H.; Hahn, D.; Schmidt, H.; Krischke, I.; Eiermann, W.; Bassermann, R.; Lissner, J.: MR Imaging of the Breast Using Gadolinium DTPA. J. Comp Ass Tomogr 10, 199-204, 1986.

Even after publication of the contrast enhancement of mammary tumors by intravenous contrast medium administration in CT, the detection sensitivity of projection mammography for iodine-containing contrast media was previously regarded as too low to be able to use this CT-detectable effect in mammography. The usability of the bromine-containing contrast media that are known as less x-ray-opaque or the metal chelate solutions that are available only in lower concentrations for this application is thus even more unlikely. Fritz, S. L.; Chang, C. H. J.; and Livingston, W. H.: (Scatter/Primary Ratios for X-Ray Spectra Modified to Enhance Iodine Contrast in Screen-film Mammography, Med Phys 10, 866-870, 1983) therefore investigate the question of whether a radiation quality that is more suitable for the absorption spectrum of the iodine can be produced by various physical measures. The results of this work still cannot be considered satisfactory, but it is believed that there is some chance for further optimization of the x-ray spectrum.

In the mid-1980's, an attempt was made to use digital subtraction angiography (DSA) with intravenous injection of contrast media. The process was not accepted since its reliability and sensitivity were too low, and in any case further testing is required (Dean, P. B.; Sickles, E. A.: Invest Radiol 20, 698-699, 1985).

The above-mentioned methods have advantages over conventional projection mammography, but also significant drawbacks such as high cost and limited availability, inadequate detection of the microlime that is important for tumor diagnosis, low spatial resolution, extended testing times, poor accessibility for biopsies, or higher radiation exposure. Although not every drawback applies to every technique, MR and, even more, CT are now used only in a very small proportion of the patients in question, and DSA is virtually not used at all for detecting mammary tumors.

Because of its almost universal availability, low cost and in many respects high performance, an improvement in the projection mammography that is introduced is therefore of great importance with respect to more reliable detection of tumors. In this respect, many tests have already been done. In particular, the recording technique and the film material that is used have been optimized over the decades; and xeroradiography has been tried and tested. New receiver systems and digitization promise further progress. Nevertheless, projection mammography, as far as can be seen now, clearly lies under the sensitivity of the best method to date, contrast-enhanced magnetic resonance tomography.

### DESCRIPTION OF THE INVENTION

It has now been found, completely surprisingly enough, that projection radiography, which is known as quite contrast medium-insensitive, can, in special cases, improve projection mammography by intravenous contrast medium administration, although the contrast media are very strongly diluted on the way through heart and lung and are not known to actively concentrate in mammary tumors.

The invention therefore relates to the use of intravenous contrast media for the production of a diagnostic agent for projection mammography.

Through the additional intravenous administration of contrast media, projection mammography achieves a sensitivity that is comparable to that of the most modern processes such as magnetic resonance tomography (MRT) while being considerably more versatile and avoiding the costs of MRT. The new process can be implemented simply and without special stress on the patients and provides a significant improvement in

a) sensitivity to the detection of focal lesions in the breast, and

b) additional information on the nature of lesions detected previously.

Its use according to the invention can be done with now available devices and agents, e.g., as follows, if the devices are operated with low radiation energy—as is common in projection mammography.

The measuring process is preferably performed as follows:
1) A normal mammogram is recorded (pre-contrast image).
2) The patient receives a commonly used urographic x-ray contrast medium at a dose of about 0.5 g to 1.5 g of. iodine/kg of body weight that is quickly injected intravenously or infused.
3) 30 seconds to 1 minute after the end of the injection, a second mammogram is recorded (post-contrast image).

Other images are optionally recorded up to about 5 minutes after the end of the injection, which, if necessary, can provide additional information on the properties of the lesion.

Devices and device settings of less than 50 kV are suitable for use according to the invention; the use of radiation that corresponds to 20 kV to 40 kV is preferred; a radiation energy of 25 kV to 35 kV is especially preferred.

For use according to the invention, all compounds are suitable that are commonly used for the production of water-soluble urographic contrast media. As examples, there can be mentioned: meglumine or lysine diatrizoate, iothalamate, ioxithalamate, iopromide, iohexol, iomeprol, iopamidol, ioversol, iobitridol, iopentol, iotrolan, iodixanol, and ioxilan (INN).

Iodine-free compounds can also be used, however, such as, e.g.:
1. Contrast media that contain bromine as an imaging element,
2. Contrast media that contain elements of atomic numbers 34, 42, 44-52, 54-60, 62-79, 82, or 83 as imaging elements,
3. Contrast media that contain chelate compounds of elements of atomic numbers 56-60, 62-79, 82, or 83 as imaging elements.

The invention therefore also relates to the use of such iodine-free compounds.

The now commonly used urographic x-ray contrast media are extremely well suited for the above-described process. It was found, surprisingly enough, that unlike in almost every other x-ray process in projection mammography, the element iodine can be exchanged completely or partially for the element bromine. This has also been discussed specifically in the past but has not proven its value in any x-ray process because of the significantly lower radiation absorption of bromine compared to iodine. In this respect, projection mammography represents an exception. It is a novel, surprising use for the compounds that are described in, e.g., EP 0 118 348 A1.

In addition, contrast media that can be excreted and are tolerable and are based on other opacifying elements, molecular and supramolecular structures are also suitable for use according to the invention.

As opacifying elements, mainly those with atomic numbers 34, 42, 44-60, 62-79, 82, or 83 are suitable. The opacifying elements can be bonded covalently to organic molecules or can be present as complexes or integrated into macromolecular structures. Substances with molecular weights of 10,000 to 80,000 D are especially advantageous. In addition, the individual contrast medium molecule components can be of larger structures, such as associates, liposomes, emulsion droplets and microparticles or nanoparticles (Parvez, Z.; Moncada, R.; Sovak, M., edts.: Contrast Media: Biological Effects and Clinical Application. Vol. III, CRC Press, Boca Raton, Fla. 1987, 73-130).

The medium is prepared in a pharmaceutically usual form in physiologically compatible vehicle media, preferably water, while using commonly used adjuvants such as stabilizers (e.g., complexes, complexing agents, antioxidants), buffers (e.g., tris, citrate, bicarbonate), emulsifiers and substances for adaptation to osmolality and electrolyte content as required.

Preferred are contrast media with concentrations of 100 mg of iodine/ml to 500 mg of iodine/ml; especially preferred are nonionic x-ray contrast media with 200 mg of iodine/ml to 400 mg of iodine/ml or a corresponding x-ray opacity when another radiation-absorbing element is selected. The agent can be administered at a dose of 150 to 1500 mg of iodine/kg of body weight (KG).

When bromine-containing compounds are used according to the invention, a concentration of 100 to 500 mg of bromine/ml in the contrast medium is preferred. The dose that can be administered is 100 to 1500 mg of bromine/kg of body weight.

When compounds of the elements of atomic numbers 34, 42, 44-52, 54-60, 62-79, 82, or 83 are used according to the invention, a concentration of 10 mmol to 2 mol/l—relative to the imaging element—in the contrast medium is preferred. The dose that can be administered is 0.1 to 2 mmol/kg of body weight (relative to the imaging element). The range of 0.2 to 0.6 mmol/kg of body weight is preferred.

When the chelate compounds of the elements of atomic numbers 56-60, 62-79, 82, or 83 are used according to the invention, a concentration of 10 mmol to 2 mol/l—relative to the imaging element—in the contrast medium is preferred. The dose that can be administered is 0.1 to 2 mmol/kg of body weight (relative to the imaging element). The range of 0.2 to 0.6 mmol/kg of body weight is preferred.

A very advantageous variant of intravenous contrast-projection mammography in the use according to the invention relates to the use of the subtraction technique, which to date has not been introduced in projection mammography. Corresponding processes have proven their value veryowell in angiography, however. In angiography, again significantly higher local iodine concentrations (in the blood) are also necessary, however, such as can be achieved in mammary tumors. In this respect, the possible use of this technique for detecting smaller lesions was not predictable. The process thus is based on the use of digital image receivers in mammography, which must have site resolution that is sufficient for this testing method. To achieve this resolution in the digital image that is necessary for mammography, it is therefore possible either to work with digital image receivers of small pixel sizes or to use digital image receivers in connection with the direct-radiographic magnification technique. Both the contrast resolution and site resolution are considerably improved by the combined use of the magnification technique with digital image receivers. As a result, it is specifically the detection of small lesions that is considerably facilitated. The process is essentially based on the following steps:
1) A normal mammogram (pre-contrast image) is recorded. The data are stored.
2) The patient receives a suitable contrast medium at a sufficient dose—quickly intravenously injected.
3) Starting at 30 seconds after the end of the injection, one or more additional mammograms are recorded and stored.
4) The data that are taken under (1) are correlated (preferably subtracted) with the data that are taken under (3), and the result is correspondingly enhanced and put out as a picture.
5) Optionally, data for speed and for the extent of the increase in contrast medium and for the kinetics of the washing process are calculated and separately visualized.

The invention therefore also relates to a device for projection mammography that is characterized by site resolution that is sufficient for the mammographic testing. This sufficient site resolution is achieved either directly via the resolution capacity of the digital image receiver or is achieved by a linkage of the digital image receiver and the direct-radiographic magnification technique. The device also contains at least one storage device for the pre-contrast image, at least one storage device for the post-contrast image, at least one computing unit for correlation (especially subtraction) of the various images, and an output device for the calculated mammogram.

Except for the correlation of the time-sequenced images or data records, it is also advantageous to correlate images that were produced with varying radiation energy. Thus, e.g., in the use of bromine-containing compounds according to the invention, an image with a radiation energy of ξ₁=35 kV and an image with a radiation energy of ξ₂25 kV can be made, and the stored images can be correlated with one another—especially subtracted from one another. In this case, suppression of the normal tissue structures in favor of the opacifying, intravenously fed element is also achieved, since the radiation absorption of the. tissue in the selected energies differs from that of the contrast medium. By repeated measurement, the time behavior-of the contrast medium concentration can also be detected and evaluated using such a device.

Another subject of the invention is therefore a device for projection mammography that is characterized by at least one storage device for an image at a radiation energy ξ₁, at least one storage device for an image at a radiation energy ξ₂, at least one computing unit for correlation of the various images, and an output device for the calculated mammogram.

In standard projection mammography, in each case only one breast is tested. To limit the necessary quantity of contrast medium, it is advantageous in the use according to the invention to test both breasts simultaneously. Devices that allow such testing are not yet known. The subjects of the invention are therefore also devices that are characterized in that they make possible simultaneous testing of both breasts.

Embodiments:

The following examples are to explain the subject of the invention without intending that it be limited to these examples.

### EXAMPLE 1

### Phantom Studies

Bismuth-, iodine- and bromine-containing contrast medium solutions ((4S)-4-(ethoxybenzyl)3,6,9-tris(carboxylatomethyl)-3,6,9-triazaundecanoic acid, bismuth complex, disodium salt, iotrolan (INN) or N-cetyl-N,N,N-trimethylammonium bromide) are produced at a concentration of 9.8 mg of Bi/ml, 6 mg of iodine/ml, or 3.8 mg of Br/ml in 2% agar. The agar gels are cut into layers that are 3 mm, 5 mm, or 10 mm thick. The contrast medium-containing gels as well as a control gel with 2.8 mg of NaCl/ml are integrated into an agar block with a thickness of 5 cm. The entire phantom is x-rayed at 28 kV and 63 mA corresponding to a mammogram, whereby the x-ray radiation in each case has to pass through about 4 cm to 5 cm of contrast medium-free agar and 3 mm to 10 mm of contrast medium-containing agar.

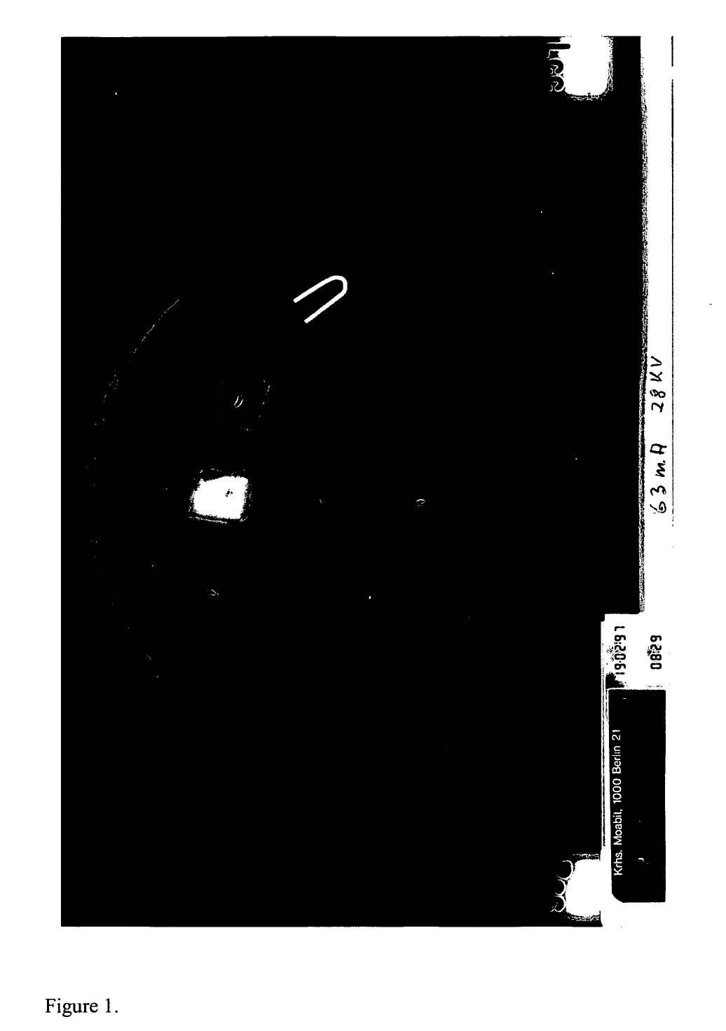

Result: Even the contrast medium-containing agar pieces that are only about 3 mm thick are readily detectable. At an equimolar concentration, bromine is, surprisingly enough, about twice as effective as iodine; bismuth is more than three times as effective as iodine (FIG. 1).

FIG. 1 shows an x-ray image at 28 kV, 63 mA of an agar phantom with embedded contrast medium-containing agar blocks of: a left series with a thickness of 5 mm, a center series with a 10 mm thickness, and a right series with a 3 mm thickness. The blocks of the upper series contain 3.8 mg of bromine/ml, those of the center series contain 6 mg of iodine/ml, and those of the lower series contain 9.8 mg of Bi/ml.

The block with NaCl is not visible.

### EXAMPLE 2

### Intravenous Contrast Medium Mammography

In a patient, a 1.5 cm×0.8 cm breast carcinoma was detected by mammography based on structures, microlime, and biopsy. Pre-operatively a check is to be made for multiple foci; in this respect, a first indwelling cannula is placed in the left arm vein (V. cubitalis) of the patient. Projection mammography is repeated before the contrast medium-is administered. Immediately after the original image, the infusion of 3 ml/kg of Ultravist®-300 (Schering AG, Berlin; active ingredient: iopromide (INN)) begins at a rate of 3 ml/sec. using an automatic injector. The first image after the administration of contrast medium is made 1 minute after the end of the infusion. The positions of the patient and the imaging device remain completely unchanged during this time, just like the imaging conditions with 28 kV of tube voltage and 63 mA.

The images after the injection of the contrast medium show a significantly enlarged area of the contrast medium image relative to the tissue that is defined as the tumor area before the administration of contrast medium, but no additional separate foci that accumulate in the breast.

1. Use of intravenous contrast media for the production of a diagnostic agent for projection mammography.

2. Use of an agent according to claim 1, characterized in that the intravenous contrast medium contains iodine as an opacifying element.

3. Use of an agent according to claim 1, wherein the intravenous contrast medium contains bromine as an opacifying element.

4. Use of an agent according to claim 1, wherein the intravenous contrast medium contains a compound of the elements of atomic numbers 34, 42, 44-52, 54-60, 62-79, 82, or 83.

5. Use of an agent according to claim 1, wherein the intravenous contrast medium contains a metal chelate of the elements of atomic numbers 56-60, 62-79, 82, or 83.

6. Use of an agent according to claim 1, wherein the intravenous contrast medium has a molecular weight of 10,000 to 80,000 D.

7. Use of an agent according to claim 1, wherein the intravenous contrast medium is present in more highly-molecular structures.

8. Use of an agent according to claim 7, wherein the intravenous contrast medium is present in the form of molecule associates, liposomes, nano- or microparticles.

9. Use of intravenous contrast media according to claim 1, wherein they are present in an x-ray opacity that corresponds to 100 mg of iodine/ml to 500 mg of iodine/ml.

10. Use of intravenous contrast media according to claim 2, wherein they are present at a concentration of 100 mg of iodine/ml to 500 mg of iodine/ml.

11. Use of intravenous contrast media according to claim 2, wherein they are administered at a dose that corresponds to 150 mg of iodine/kg to 1500 mg of iodine/kg of body weight.

12. Use of intravenous contrast media according to claim 3, wherein they are present at a concentration of 100 mg of bromine/ml to 500 mg of bromine/ml.

13. Use of intravenous contrast media according to claim 3, wherein they are administered at a dose that corresponds to 100 mg of bromine/kg to 1500 mg of bromine/kg of body weight.

14. Use of intravenous contrast media according to claim 4, wherein they are present at a concentration of 10 mmol-2 mol/l.

15. Use of intravenous contrast media according to claim 4, wherein they are administered at a dose of 0.1-2 mmol/kg of body weight.

16. Use of intravenous contrast media according to claim 5, wherein they are present at a concentration of 10 mmol/l-2 mol/l.

17. Use of intravenous contrast media according to claim 5, wherein they are administered at a dose of 0.1-2 mmol/kg of body weight.
